(19) United States
(12) Patent Application Publication
(10) Pub. No.: US 2022/0133433 A1
(43) Pub. Date: May 5, 2022

(54) Zygomatic Dental Implant Guide and Method of Use

(71) Applicant: Joseph Amalfitano, Traverse City, MI (US)

(72) Inventor: Joseph Amalfitano, Traverse City, MI (US)

(21) Appl. No.: 17/090,232

(22) Filed: Nov. 5, 2020

(51) Int. Cl. A61C 1/08; A61C 8/00

(52) U.S. Cl. CPC A61C 1/084; A61C 8/0089

(57) ABSTRACT

A zygomatic implant guide apparatus includes a first surgical guide conforming to a patient's maxillary region, and a second surgical guide that also conforms to the patient's maxillary region. The first and second surgical guides include maxillary sinus windows, and the second surgical guide includes at least one bushing sleeve. A bushing is provided for insertion into the bushing sleeve, extending into the patient's maxillary sinus in order to engage and govern the position of a zygomatic implant drill as it travels through the patient's maxillary sinus to engage the patient's zygomatic bone.

### BACKGROUND

### Field of the Invention

The present invention generally relates to dental implants. More specifically, the present invention relates to a method and apparatus for guiding the installation of a zygomatic dental implant

### Related Art

Zygomatic dental implants are known in the art. Current apparatus and techniques for installing implants in a patient's zygoma, due to maxillary bone loss or other reasons, incur many risks. The most significant risks in conventional zygomatic implants are orbital penetration or improper insertion in the infra temporal fossa. Extra-sinus approaches have been developed, which are easier to perform and may avoid common risks inherent in zygomatic implant procedures, but extra-sinus procedures lose any opportunity for crestal engagement of the zygomatic implant. Additionally, not all implants can, or should be extra-maxillary. Ideally, a prosthetic position should be located that is not too far in a palatal direction and allows the prosthetic screw to emerge near the occlusal surface of the planned tooth reconstruction. Some patients may require a completely intra-sinus approach, while others may need a completely extra sinus approach, and several configurations are possible between these two extremes. Thus there is a need to treat all of these zygoma presentations. In most instances, a screw access hole should penetrate between the second premolar and the first molar, and then match with the best possible bone engagement in the zygoma.

Hence, what is needed is a zygomatic implant apparatus and method that provides effective guidance from the crestal region to the zygoma, and which does so in a manner avoiding injury to surrounding tissues. Also needed is a zygomatic implant apparatus and method that is customizable for a particular patient, and functions in procedures where other implants may be installed without the limitations of existing techniques.

### SUMMARY

A zygomatic implant guide apparatus for guiding the installation of a zygomatic dental implant is provided. The apparatus includes a first surgical guide configured to conform to at least a portion of a patient's maxillary arch, and at least a portion of an area below the patient's maxillary sinus. The first surgical guide is preferably further configured to have a first maxillary sinus window, the first maxillary sinus window being oriented on the lateral wall of the sinus in the trajectory of the planned zygoma implant. A second surgical guide is also provided. Like the first surgical guide, the second surgical guide is configured to conform to at least a portion of a patient's maxillary arch, and at least a portion of an area below the patient's zygoma. The second surgical guide further configured with a second maxillary sinus window and at least one bushing sleeve.

At least one bushing is provided and configured for insertion into the bushing sleeve. The bushing is also configured to extend into the patient's maxillary sinus in order to engage and govern the position of a zygomatic implant drill as it travels through the patient's maxillary sinus and engages the zygoma, thereby aligning the zygomatic implant drill such that it enters a zygomatic bone in the patient in a proper position.

In various embodiments of the apparatus, the first maxillary sinus window may be centered over the trajectory of the planned zygoma drills. Preferably the second maxillary sinus window is also centered over the planned trajectory of the zygoma drills. The bushing sleeve is preferably located adjacent the second maxillary sinus window guide opening, with other bushing sleeves disposed in other locations on the second surgical guide. In one embodiment, the second surgical guide may include two bushing sleeves located adjacent the second maxillary sinus window and adjacent the patient's maxillary arch, respectively.

Preferably all bushings are configured to seat in their respective bushing sleeves in a pressure fit. In a preferred embodiment, the bushing comprises an insertion body configured to extend beyond the bushing sleeve, extending into the patient's maxillary sinus. The bushing may include a flange which is configured to engage the bushing sleeve, thereby ensuring it's proper location in the patient's maxillary sinus. The bushing may have a first hole oriented in parallel to the zygomatic implant drill, and through which the zygomatic implant drill passes. Alternatively, the bushing may have a concave surface in lieu of the first hole, the concave surface configured to partially surround the zygomatic implant drill and guide it along a predetermined course through the patient's maxillary sinus.

The bushing may also have a second hole proximal the flange, the second hole for accommodating a floss-like tether in order to retrieve the bushing if disengaged. The second surgical guide may also include a pterygoid implant bushing.

A method of guiding a zygomatic implant drill during installation includes the following steps: A first surgical guide is provided. The first surgical guide is shaped to conform to at least a portion of a patient's maxillary arch, and also conform to at least a portion of an area below the patient's zygoma. The first surgical guide is also formed with a first maxillary sinus window. A second surgical guide is also provided. Like the first surgical guide, the second surgical guide is shaped to conform to at least a portion of the patient's maxillary arch, and conform to at least a portion of the area below the zygoma. The second surgical guide includes a second maxillary sinus window and at least one bushing sleeve. A bushing is provided, and configured for insertion into the at least one bushing sleeve, and to engage and govern the position of a zygomatic implant drill.

The method may also include the steps of placing the first surgical guide against at least a portion of the patient's maxillary arch and the bone of the lateral sinus wall, and marking a maxillary window position below the patient's zygoma through the first surgical guide. A maxillary window is cut at the maxillary window position for access to the patient's maxillary sinus. The first surgical guide is removed, and the second surgical guide is affixed against at least a portion of the patient's maxillary arch and the area below the zygoma. This surgical guide may be affixed to the bone structure using one or two or more threaded surgical screws in areas not impinging the planned zygoma implant trajectory. The bushing is inserted into the bushing sleeve, such that the bushing extends into the patient's maxillary sinus, and a zygomatic dental drill extends through the bushing, wherein the bushing guides the zygomatic dental drill to a predetermined position in the patient's zygomatic bone.

As preliminary steps, the method may also include scanning a maxillary region of the patient, preparing a model of the patient's scanned maxillary region determining an optimal placement position of a zygomatic implant, orienting the at least one bushing in a first position that engages and governs the position of a zygomatic implant drill, and orienting the at least one bushing sleeve in a second position complimentary to the first position, with the at least one bushing securely anchored therein. In one embodiment, a flange may be formed on the bushing, with the flange oriented such that it engages the at least one bushing sleeve. Additionally, a bushing sleeve may be formed adjacent the second maxillary sinus window.

### BRIEF DESCRIPTION OF THE FIGURES

FIG. 1 illustrates a scan of a patient's maxillary region including the zygoma;

FIG. 2 illustrates a computer modeled first surgical guide, including a maxillary sinus window guide used to mark the location of a maxillary sinus window;

FIG. 3 illustrates a computer modeled second surgical guide, including supports for an intra sinus bushing and an alveolar bushing;

FIG. 4 illustrates a 3D printed first surgical guide;

FIG. 5 illustrates a 3D printed second surgical guide;

FIG. 6 illustrates a full drill bushing for use as an intra sinus bushing or an alveolar bushing;

FIG. 7 illustrates a half drill bushing for use as an intra sinus bushing or an alveolar bushing; and

FIG. 8 illustrates a bushing support sleeve for holding an intra sinus bushing or an alveolar bushing in position.

FIG. 9 illustrates a pterygoid implant for insertion into the second surgical guide;

FIG. 10 illustrates an exemplary scan body;

FIG. 11 illustrates a series of scan bodies in place for a double zygomatic implant;

FIG. 12 illustrates a sinus window guide for a double zygomatic implant;

FIG. 13 illustrates a double zygomatic implant guide with bushing sleeves in place;

FIG. 14 illustrates a double zygomatic implant guide in place on a right side zygomatic bone.

### REFERENCE NUMBERS

10. maxilla
12. zygoma
14. maxillary sinus region
16. tertiary maxilla scan
18. alveolar bushing
20. intra sinus bushing
22. first surgical guide
24. intra sinus bushing site
26. alveolar bushing site
28. pterygoid bushing site
30. maxillary sinus window guide
32. second surgical guide
34. intra sinus bushing sleeve
36. alveolar bushing sleeve
38. pterygoid implant bushing
40. full bushing
42. intra sinus bushing
44. alveolar bushing
46. insertion body
48. flange
50. handle portion
52. first hole
54. second hole
56. half bushing
58. bushing sleeve
60. bushing portal
62. bushing lip
64. scan body
66. pterygoid bushing sleeve
68. scan body guide hole
70. first double zygomatic implant guide
72. first maxillary sinus window guide
74. second maxillary sinus window guide
76. second double zygomatic implant guide
78. first intra sinus bushing site
80. second intra sinus bushing site
82. first alveolar bushing site
84. second alveolar bushing site
86. first intra sinus bushing sleeve
88. second intra sinus bushing sleeve
90. first alveolar bushing sleeve
92. second alveolar bushing sleeve

### DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

Overview

An improved method for preparing and installing zygomatic dental implants is provided, including a customized zygomatic dental implant guide apparatus. A patient's maxilla and zygoma region is first three-dimensionally scanned (e.g., using cone beam computed tomography or similar technology). From the scan, a series of apparatus are created, including a first surgical guide 22, a second surgical guide 32, and a series of bushings including an intra sinus bushing 42 and an alveolar bushing 44. An intra sinus bushing sleeve 34 and an alveolar bushing sleeve 36 are incorporated into the second surgical guide 32 for holding the bushings, and to create a drilling guide for a surgical drill in advance of implantation and fixation of zygomatic implants. The method of use includes a first phase in which the guide apparatus and bushings are created, and a second phase in which these apparatus are used to install a zygomatic implant.

In the apparatus creation phase of the method, a 3D initial maxilla scan is used to create a 3D printed model of the patient's maxilla and zygoma region. Using a drill kit on the 3D printed model, the optimal trajectory of the zygomatic implant is determined, first starting with ideal prosthetic positioning on the alveolus and then ensuring deep penetration and adequate anchorage of the implant in the zygoma. After incorporating scan bodies into the 3D printed model, the model is scanned and bushings for guiding a surgical drill are put in place. This augmented model is then computer modeled, and the first surgical guide 22 and second surgical guide 32 are computer modeled and 3D printed.

The first surgical guide 22 is configured for placement on the maxillary bone, and includes a maxillary sinus window guide 30 for marking the position of a maxillary sinus window. The second surgical guide 32 is configured with bushing supports to hold the intra sinus bushing 42 and alveolar bushing 44 in a position for guiding the zygomatic drill. Optionally, guide tubes for pterygoid implant placement can be incorporated into the same guide. The guide sleeves for the pterygoid implant have been designed to allow for the use of osteotomes, which are beneficial due to the soft nature of bone in this region of the maxilla.

In the surgery phase of the method, the first surgical guide 22 is placed on the lateral wall of the patient's maxillary sinus, such that it also preferably covers a portion of the patient's maxillary arch. Using the maxillary sinus window guide 30, the position of the maxillary sinus window is marked, and when created, will allow visual access to the patient's maxillary sinus interior. Once the first surgical guide 22 is removed and the maxillary sinus window created, the second surgical guide 32 is placed in position and fixed in place on the lateral wall of the patient's maxillary sinus. The second surgical guide 32 accepts an intra sinus bushing 42 and an alveolar bushing 44, which guide a zygomatic drill through the crestal region, across maxillary sinus, and into the zygoma. Once the zygomatic drill establishes the proper trajectory and placement of a zygomatic implant, the second surgical implant 32 and bushings are removed along with the surgical guide and the zygomatic implant is installed, anchored in the maxilla and zygoma according to practice. This process ensures accurate placement of the zygomatic implant without risking injury to surrounding areas.

Detailed Description of Apparatus Preparation

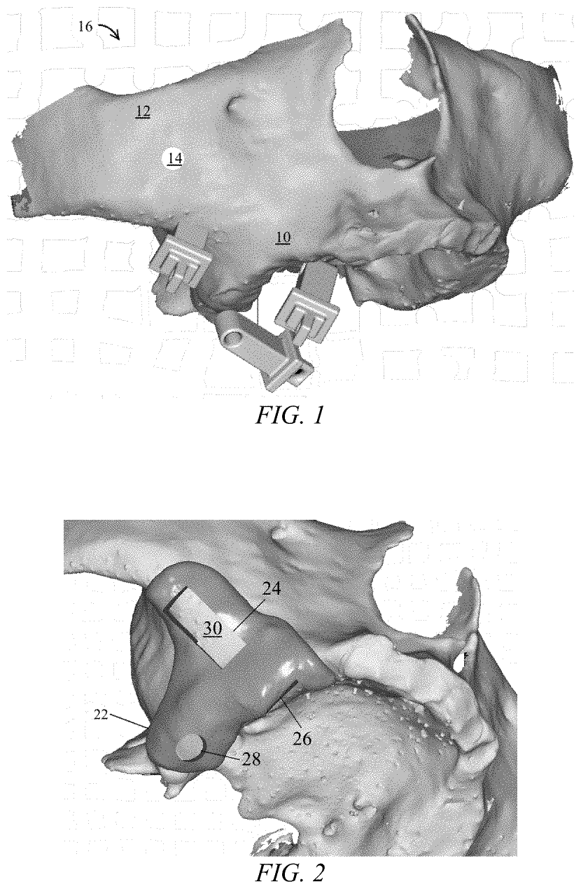

Referring to the FIG. 1, the first step in an improved method and apparatus for preparing and installing a zygomatic implant is creating a CBCT (cone beam computed tomography) or similar three-dimensional scanned image of the patient's pterygoid region, including the maxilla 10, zygoma 12 and maxillary sinus region 14, referred to herein as an initial maxilla scan. The initial maxilla scan is saved as a base scan to be referred to in later stages of the process. A copy of the initial maxillary scan is made.

Thereafter, in the copy of the initial maxilla scan, extraneous (i.e., non-maxillary, non-zygomatic) bone and other structures that were captured in the initial maxilla scan are segmented out (i.e., removed), including the patient's teeth and any pre-existing implants, the copy is saved as a secondary maxilla scan. The segmentation process may be accomplished more quickly by ‘thresholding’, i.e., adjusting the scan threshold to segment out non-bone structures in the scan, which may include soft tissue and hard tissue, including during the process of manually removing tooth and pre-existing implant scans. Optimal placement of the zygoma implant is based on the secondary maxilla scan:

In one embodiment, the secondary maxilla scan and supporting scans are imported into 3D design software, enabling them to be visually manipulated, to determine the ideal position of the zygomatic implant. Preferably, this accomplishes maximized bone contact in the zygoma for secure anchorage. In an alternative embodiment, a physical model may be 3D printed to create a physical representation of the secondary maxilla scan. A test surgical kit and various drills may then be used on the physical model, on a lab bench for example, to locate the zygomatic implant physically in an ideal position, thereby maximizing bone contact and secure zygomatic implant anchorage.

When a physical model is employed, the user preferably drills through the alveolar ridge on the model, through the maxillary sinus, and into the zygoma such that the drill represents the position where the zygomatic implant will be. If the initial drill position is sub-optimal, the user may readjust the position of the drill to find an ideal zygoma insertion position. In the process, scan bodies are placed on the implant site to identify the position of the zygomatic implant when the implant bearing physical model is scanned. The implant bearing physical model is then sprayed with a scanning spray and the model, including the scan bodies is 3D scanned to create a tertiary maxilla scan.

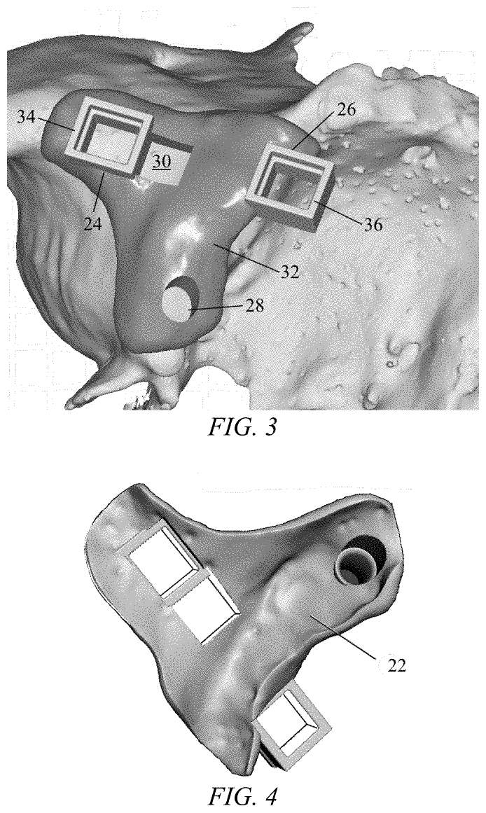

When the implant bearing physical model is scanned using a lab quality scanner, the resulting STL data is imported into surgical guide design software. The STL files of the pretreatment scanned maxilla (i.e., the primary maxilla scan and the secondary maxilla scan) and printed maxilla, are then aligned with the scan of the implant bearing physical model to show a uniform scan of the zygomatic process and maxillary arch with the zygomatic implant in position. A first surgical guide 22 (FIG. 2) and a second surgical guide 32 (FIG. 3) may be created using 3D design software.

Referring to FIG. 2, a maxillary scan is shown with the first surgical guide 22 in position at the implant site. As discussed, the first surgical guide 22 may be created in free form using 3D design software. In any desired form, the first surgical guide 22 preferably covers a portion the maxillary arch and the lateral wall of the maxillary sinus, and is configured for locating the intra sinus bushing site 24 and the alveolar bushing site 26. The first surgical guide 22 is preferably designed on the untreated first maxilla scan to ensure a proper conforming fit against the patient's maxillary arch and below the patient's zygoma.

Space is created at the intra sinus bushing site 24, where the intra sinus bushing 42 (FIG. 1) will penetrate the patient's maxillary sinus, and the alveolar bushing site 26, to be occupied by the alveolar bushing 44 (FIG. 1). Space is removed from the first surgical guide 22, including space for accommodating the intra sinus bushing sleeve 34 (FIG. 3) that will hold the intra sinus bushing 42 and the alveolar bushing sleeve 36 (FIG. 3) that will hold the alveolar bushing 44 in place. A pterygoid implant bushing site 28 may also be created if desired where a pterygoid implant bushing 38 will be installed. A maxillary sinus window guide 30 is created in the first surgical guide 22 for marking the location of a maxillary sinus window, which will be created in the patient and used to view the zygomatic drill as it travels through the maxillary sinus, thereby verifying the correct position of a surgical drill as it passes through the maxillary sinus, and intra sinus bushing 42, when the drill is inserted through the intra sinus bushing 42 and into the zygomatic bone. Once properly formed and conforming against the maxillary scan, the first surgical guide 22 can be exported and 3D printed for use on the patient.

Referring to FIG. 3, once the first surgical guide 22 has been saved, exported, and 3D printed, a second surgical guide 32 is created based on the first surgical guide 22 by adding an intra sinus bushing sleeve 34 to the maxillary sinus window guide 30, for supporting the intra sinus bushing 42 in its proper position. An alveolar bushing sleeve 36 is added to the alveolar bushing site 26 for supporting the alveolar bushing 44. Depending on the procedure, other sleeves may be added, such as for a pterygoid implant or other conventional implants. This procedure allows zygomatic implants and pterygoid implants to be installed during the same procedure. Once the intra sinus bushing sleeve 34, alveolar bushing sleeve 36, and any additional sleeves, such as a pterygoid implant bushing 38 (FIG. 9) are added, the resulting second surgical guide 32 may be exported and 3D printed.

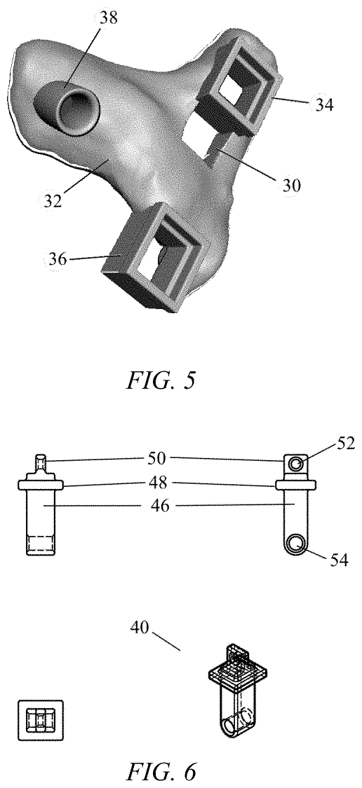

Referring to FIGS. 4 and 5, the first surgical guide 22 and the second surgical guide 32 are shown exported and 3D printed. In various embodiments the first surgical guide 22 and the second surgical guide 32 may be created by milling, 3D printing, laser sintering, or similar 3D production method in preparation for installation on the patient. The first surgical guide 22 and the second surgical guide 32 are first test fit on the physical model. They may also be test fit on a model created from the non-manipulated initial maxilla scan (a ‘non-manipulated model’) of the patient by installing the intra sinus bushing 42 and the alveolar bushing 44 and drilling the nonmanipulated model to ensure proper zygomatic drill trajectory (and optionally, pterygoid or other implant drill trajectory if using a pterygoid bushing). When testing the fit of the first surgical guide 22 and the second surgical guide 32, a user should verify that the surgical drill tooling fits passively in the first bushing, second bushing and other bushings (e.g., pterygoid bushing, etc.) to ensure impingement of the surgical drill will not occur.

Referring to FIG. 6, a full bushing 40 is shown, which may be used interchangeably as either the intra sinus bushing 42 (when inserted into the intra sinus bushing sleeve 34), or the alveolar bushing 44 (when inserted into the alveolar bushing sleeve 36). The full bushing 40 includes an insertion body 46, a flange 48 and a handle portion 50. The handle portion 50 includes a first hole 52 for accepting dental floss, or a similar retaining tether to retrieve the full bushing 40 if it inadvertently falls into the recesses of a patient's mouth. The insertion body 46 includes a second hole 54 through which a surgical drill passes. The second hole 54 is therefore axially aligned with, and guides the surgical drill toward the appropriate drilling location in the zygomatic bone.

The flange 48 is sized to engage either the intra sinus bushing sleeve 34 or the alveolar bushing sleeve 36. Preferably the insertion body 46 of the full bushing 40 engages either the intra sinus bushing sleeve 34 or the alveolar bushing sleeve 36 in a pressure fit to hold it in place. In various additional embodiments, a variety of alveolar bushings 44 and intra sinus bushings 42 having second holes 54 of varying sizes may be produced and used according to the various diameters of drills in the drill kit used for a given procedure.

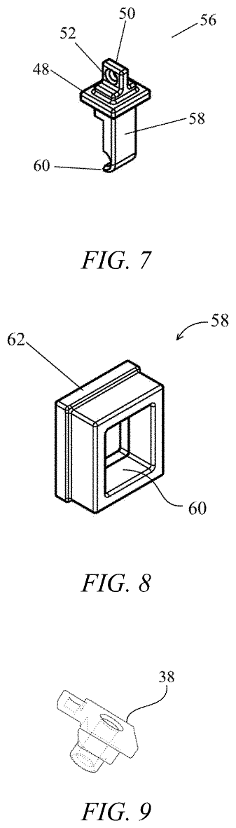

Referring to FIG. 7, a half bushing 56 is shown, which also may be used as the intra sinus bushing 42 or alveolar bushing 44, and may be inserted into the intra sinus bushing sleeve 34 or alveolar bushing sleeve 36 in place of the full bushing 40. Like the full bushing 40, the half bushing 56 includes a flange 48, and a handle portion 50 with a first hole 52 for accommodating floss or a floss-like tether. Unlike the full bushing 40, the half bushing 56 has a half insertion body 58, and terminates in a semi-circular concave guide 60 rather than a second hole 54. The half bushing 56 may be used in place of the full bushing 40 when there is a risk of impingement of the surgical drill, which may be detected in the software or working on the physical model. Referring to FIG. 8, an exemplary support sleeve 62 is shown, which may be incorporated into the second surgical guide 32 as the intra sinus bushing sleeve 34 or the alveolar bushing sleeve 36.

Referring to FIG. 8, a bushing sleeve 58 is shown. The bushing sleeve 58 is sized for accommodating the intra sinus bushing 42 and/or alveolar bushing 44, and incorporation into the second surgical guide 32. The bushing sleeve 58 includes a bushing portal 60, preferably sized to accommodate the intra sinus bushing 42 and/or alveolar bushing 44 in a pressure fit. The bushing sleeve 58 also preferably includes a bushing lip 62 for engaging the flange 48 of the full bushing 40 and/or the half bushing 56. The bushings may be manufactured in different lengths depending on design factors of the surgical guide.

Referring to FIG. 9, a pterygoid implant bushing 38 is shown for insertion into the second surgical guide. The pterygoid implant bushing 38 functions similar to the other bushings, in that it guides a pterygoid drill into the patient's pterygoid region for proper positioning of the pterygoid implant. The pterygoid bushing can be configured to accept either surgical drills or ostotomes for creating the implant osteotomy.

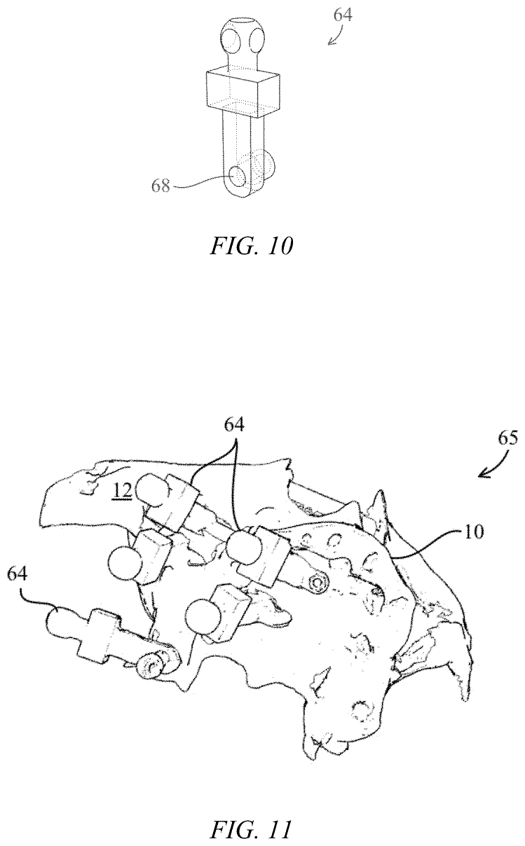

Referring to FIG. 10, a scan body 64 is shown. Scan bodies 64 are used for locating the positions of the intra sinus bushsing sleeve 34, the alveolar bushing sleeve 36, and optionally, a pterygoid bushing sleeve 66. One or more scan bodies 64 allow for a digital implant impression on a scanned model or a 3D computer model. Preferably, the scan body 64 is substantially the same size as a bushing, including a scan body guide hole 68 identifying the axis along which a dental drill will pass during surgery. By orienting each of the various busing sleeves such that they conform to the a scan body position, the correct position of a bushing sleeve, and thus a bushing is ensured.

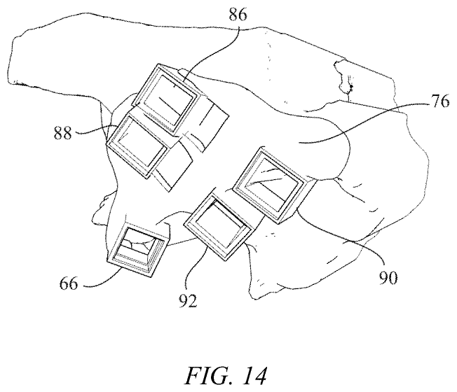

Referring to FIGS. 11-14, in certain implementations, a double zygomatic implant guide 64 may be created and used, if necessary. Referring to FIG. 11, like the single implant model discussed above, a scan or model of the patient's zygomatic 12 and maxillary 10 region are created, and scan bodies 64 are placed in position to identify the location of bushing sleeves (FIGS. 13-14) around which a first double zygomatic implant guide 70 will be created. The model may then be scanned. If a 3D model is used, the model may simply be manipulated by computer.

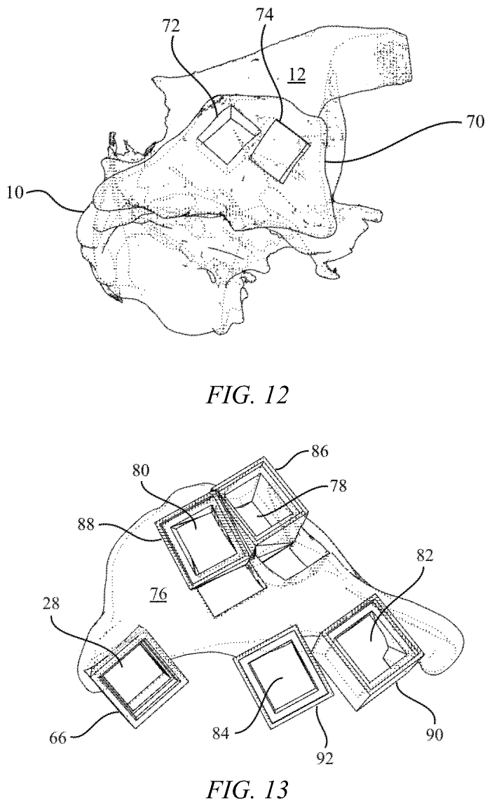

Referring to FIG. 12, a first double zygomatic implant gude 70 is shown with a first maxillary sinus window guide 72 and a second maxillary sinus window guide 74 in position for installing two zygomatic implants.

Referring to FIG. 13, a second double zygomatic implant guide 76 is shown, ready for fixation in patient. The second double zygomatic implant guide 76 includes a first intra sinus bushing site 78 and a second intra sinus bushing site 80, a first alveolar bushing site 82 and a second alveolar bushing site 84, and a pterygoid implant bushing site 28. A first intra sinus bushing sleeve 86 and a second intra sinus bushing sleeve 88 are installed at the first intra sinus bushing site 78 and the second intra sinus bushing site 80, respectively. A first alveolar bushing sleeve 90 and a second alveolar bushing sleeve 92, are installed at the first alveolar bushing site 82 and the second alveolar bushing site 84, respectively. A pterygoid bushing sleeve 66 has been installed at the pterygoid bushing site 28.

Referring to FIG. 14, the second double zygomatic implant guide 76 is shown installed on a patient, after two maxillary sinus windows are created using the first maxillary sinus window guide 72 and the second maxillary sinus window guide 74, and ready for the installation of bushings for each of two zygomatic implants, as well as a pterygoid implant.

In preparing for a surgical implant procedure, once the first surgical guide and second surgical guide (either single or double), first bushing, second bushing, and optional components, such as the pterygoid bushing are created, they are autoclaved or sterilized by other means for surgery. Immediately prior to an implant procedure, floss or a similar tethering apparatus is installed in the first hole of the handle potion first bushing and the second bushing, so they may be installed and easily removed prior to installation of the zygomatic implant.

Detailed Description of a Zygomatic Implant Guide Protocol

Once an appropriate maxillary scan has been obtained, including 3D printing the maxillary scan (or through 3D manipulation), the best trajectory of the zygomatic implant is ascertained, which governs the optimal position of the intra sinus bushing 42 and the alveolar bushing 44. Thereafter the first surgical guide 22 with the maxillary sinus window guide 30, and the second surgical guide 32 with the intra sinus bushing sleeve 34 and the alveolar bushing sleeve 36 are prepared. In the process, as discussed above, the model maxilla may be re-drilled to locate the optimal position of the zygomatic implant. The first surgical guide 22 and the second surgical guide 32, along with appropriate full bushings 40 and or half bushings 56, are 3D printed and sterilized, and an appropriate zygomatic implant prepared for installation.

To install a zygomatic implant the first surgical guide 22 is fitted in place. The first surgical guide 22 may be used to assist with reflection or elevation of the mucoperiosteal flap and placement of an appropriate zygomatic retractor by identifying the direction of the zygomatic notch. Once the first surgical guide 22 is properly seated in place, a sinus slot window mark is created using a sterile pencil or marker to trace the maxillary sinus window guide 30. The first surgical guide 22 is then removed and surgical burs are used to create a maxillary sinus window in the patient, opening up the interior of the patient's maxillary sinus to view. In the process, the sinus membrane may be reflected according to practice.

Once the maxillary sinus window created, the second surgical guide 32 is placed in position, and at least two pilot holes (not shown) are created using a small pilot drill. Thereafter, two self-tapping screws are drilled into the maxilla to anchor the second surgical guide 32 in place. In one preferred embodiment, the self-tapping screws are approximately 1.5 mm diameter and 12 mm long. With the second surgical guide 32 sufficiently anchored in position, full bushings 40 and/or half bushings 56 are selected and installed in position to form an intra sinus bushing 42 and an alveolar bushing 44. Prior to their installation, floss or a similar tethering material is inserted into the first hole 52 in the handle portion 50 of each bushing, for retrieval if necessary.

The alveolar bushing 44 is inserted into the alveolar bushing sleeve 36, and the intra sinus bushing 42 is inserted into the intra sinus bushing sleeve 34, such that the flange 48 of each bushing is flush against each support sleeve and the alveolar bushing 44 and the intra sinus bushing 42 are each held in the second surgical guide 32 under a pressure fit, with the insertion body 46 (or half insertion body 58) of the intra sinus bushing 42 extending inside the patient's maxillary sinus and visible through the maxillary sinus window. Despite ensuring the appropriate intra sinus bushing 42 angle when preparing the second surgical guide 32, if drill impingement appears likely, a full bushing 40 may be easily exchanged for a half bushing 56 during surgery.

In preparing for a zygomatic implant, a series of intra sinus bushings 42 (and alveolar bushings 44) of different sizes corresponding to differing drill diameters in a particular surgical kit may be used. Various lengths of bushings may be required based on the depth of the zygoma implant trajectory within the sinus cavity and position of the alveolar bone. For example, a 2.7 mm drill, then a 2.9 mm drill, then a final drill may be used to create a pilot hole in the zygoma, with multiple alveolar and intra sinus bushings having guide holes of complimentary size used accordingly. With the intra sinus bushing 42 and the alveolar bushing 44 in position, a drill (or series of drills) are inserted through the alveolar bushing and maxilla, through the maxillary sinus, and into the zygoma. During this process, the drill may be observed through the maxillary sinus window to ensure it engages the intra sinus bushing 42 properly. The drill is inserted until its apical end perforates the zygomatic bone.

Once the drilling sequence is complete, the second surgical guide 32, along with the intra sinus bushing 42 and alveolar bushing 44, is removed. The maxillary alveolar crestal region is prepared for an individual zygoma implant of appropriate length and diameter, and the zygoma implant is installed according to conventional techniques, using the maxillary sinus window to view the zygomatic implant as it is installed. Using the first surgical guide 22 and second surgical guide 32, along with the intra sinus bushing 42 and alveolar bushing 44, the apparatus can be adapted to any surgical zygoma kit and may be used alongside conventional implants when multiple implants, including zygomatic implants, are to be installed in the same procedure. By using a second surgical guide 32 that is fixed in position, the design does not require a separate holding apparatus that conventional zygoma implant guides use to support an implant drill.

For purposes of discussion, a right-side zygomatic implant is shown and discussed herein. It should be understood that a left-side zygomatic implant may be installed using the current method and apparatus in a similar manner, reoriented for a left-side installation.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. A zygomatic implant guide apparatus for zygomatic dental implants, the apparatus comprising:
a first surgical guide configured to conform to at least a portion of a patient's maxillary arch, and at least a portion of an area lateral to the maxillary sinus; the first surgical guide further configured with a first maxillary sinus window; a second surgical guide configured to conform to at least a portion of a patient's maxillary arch, and at least a portion of an area zygoma; the second surgical guide further configured with a second maxillary sinus window and at least one bushing sleeve; at least one bushing configured for insertion into the at least one bushing sleeve, the bushing further configured to engage and govern the position of a zygomatic implant drill; wherein the at least one bushing is configured to extend into the patient's maxillary sinus when engaged with the at least one bushing sleeve, thereby aligning the zygomatic implant drill such that it enters a zygomatic bone in the patient in a predetermined position.

2. The apparatus of claim 1, wherein the first maxillary sinus window guide is configured for placement on the lateral wall of the maxillary sinus.

3. The apparatus of claim 1, wherein the second maxillary sinus window guide is configured for placement on the lateral wall of the maxillary sinus.

4. The apparatus of claim 1, wherein the at least one bushing sleeve is located adjacent the second maxillary sinus window guide opening.

5. The apparatus of claim 1, wherein the second surgical guide comprises two bushing sleeves located adjacent the second maxillary sinus window and adjacent the patient's maxillary arch, respectively.

6. The apparatus of claim 1, wherein the at least one bushing is configured to seat in the at least one bushing sleeve in a pressure fit. The apparatus of claim 1, wherein the at least one bushing comprises an insertion body configured to extend beyond the bushing sleeve.

8. The apparatus of claim 1, wherein the at least one bushing comprises a flange configured to engage the at least one bushing sleeve.

9. The apparatus of claim 1, wherein the at least one bushing comprises a first hole oriented in parallel to the zygomatic implant drill.

10. The apparatus of claim 1, wherein the at least one bushing comprises a concave surface configured to partially surround the zygomatic implant drill.

11. The apparatus of claim 1, wherein the at least one bushing comprises a second hole proximal a flange for engaging the at least one bushing sleeve.

12. The apparatus of claim 1, wherein the second surgical guide comprises a pterygoid implant bushing.

13. A method of guiding a zygomatic implant drill during installation, the method comprising the steps of:
providing a first surgical guide configuring the first surgical guide to conform to at least a portion of a patient's maxillary arch, and conform to at least a portion of an area below the patient's maxillary sinus; configuring the first surgical guide with a first maxillary sinus window; providing a second surgical guide; configuring the second surgical guide to conform to at least a portion of the patient's maxillary arch, and conform to at least a portion of the bone on the laterial wall of the maxillary sinus; configuring the second surgical guide with a second maxillary sinus window and at least one bushing sleeve; providing at least one bushing; and configuring the at least one bushing for insertion into the at least one bushing sleeve, and to engage and govern the position of a zygomatic implant drill.

14. The method of claim 13, further comprising the steps of:
placing the first surgical guide against the at least a portion of the patient's maxillary arch and the area below the patient's maxillary sinus; marking a maxillary window position below the patient's maxillary sinus through the first surgical guide; cutting a maxillary window at the maxillary window position; removing the first surgical guide, and affixing the second surgical guide against the at least a portion of the patient's maxillary arch and the bone lateral to the maxillary sinus; inserting the at least one bushing through the at least one bushing sleeve, such that the bushing extends into the patient's maxillary sinus; and inserting a zygomatic dental drill through the at least one bushing, wherein the bushing guides the zygomatic dental drill to a predetermined position in a zygomatic bone of the patient.

15. The method of claim 13, further comprising the steps of:
scanning a maxillary region of the patient; preparing a model of the patient's scanned maxillary region; determining an optimal placement position of a zygomatic implant; orienting the at least one bushing in a first position that engages and governs the position of a zygomatic implant drill; and orienting the at least one bushing sleeve in a second position complimentary to the first position, with the at least one bushing securely anchored therein.

16. The method of claim 13, further comprising the step of forming a flange on the at least one bushing, and orienting the flanch such that it engages the at least one bushing sleeve.

17. The method of claim 13, further comprising the step of forming the at least one bushing sleeve adjacent the second maxillary sinus window.
